(19) United States
(12) Patent Application Publication
(10) Pub. No.: US 2024/0407357 A1
(43) Pub. Date: Dec. 12, 2024

(54) Microencapsulation

(71) Applicant: SYNGENTA CROP PROTECTION AG, Basel (CH)

(72) Inventors: Philip TAYLOR, Bracknell, Berkshire (GB); Christopher Ian LINDSAY, Bracknell, Berkshire (GB)

(73) Assignee: SYNGENTA CROP PROTECTION AG, Basel (CH)

(21) Appl. No.: 18/699,670

(22) PCT Filed: Oct. 10, 2022
(86) PCT No.: PCT/EP2022/078027

§ 371 Date: Apr. 9, 2024

(30) Foreign Application Priority Data
Oct. 15, 2021 (GB) 2114759.0

(51) Int. Cl. A01N 25/04; A01N 25/28; A01N 55/00; A01N 59/06; A01P 3/00; A01P 17/00; A01P 21/00; B01J 13/08

(52) U.S. Cl. CPC A01N 25/04; A01N 25/28; A01N 55/00; A01N 59/06; A01P 3/00; A01P 17/00; A01P 21/00; B01J 13/08

(57) ABSTRACT

A method of forming a Pickering emulsion, comprising the combination of an aqueous phase and an oil phase, wherein the aqueous phase comprises clay particles; and wherein the oil phase comprises tetraalkyl orthosilicate.

The present invention relates to a novel process for making microcapsules and to microcapsules made by the process. It also relates to a process for the use of the microcapsules.

Microcapsules are small capsules which comprise a wall which surrounds an encapsulated material, generally a liquid. They may be used for protecting the encapsulated material from the external environment, for example from degradation by air or light. They may also be used to isolate hazardous materials within the microcapsule to make them safer to handle or use. Microcapsules are known to be used for agrochemicals, particularly insecticides such as lambda cyhalothrin, to protect them from degradation by UV light and to provide controlled release following application.

Certain known microcapsules are made by interfacial polymerisation. In such a process a solution is first formed of a first monomer, such as a polyisocyanate, in a water-insoluble liquid to be encapsulated. The solution may also contain a biologically active ingredient. This solution is then dispersed in water together with surfactants to form an emulsion. A suitable second monomer such as a polyamine is added to the water and this reacts with the first monomer at the surface of the emulsion droplets to make a cross-linked polymer, in this example a polyurea, which forms a microcapsule wall around the droplets. Known first and second monomers also include polyisocyanate and polyol to make a polyurethane wall, polyfunctional acid halide and polyamine to make a polyamide wall and polyfunctional acid halide and polyol to make a polyester wall.

However, the use of microcapsules formed from organic monomers and/or cross-linkers is undesirable due to their poor biodegradability and classification as micro-plastics. There is therefore a pressing need for a method of microencapsulation that addresses these problems.

In a first aspect of the present invention there is therefore provided a method of forming a Pickering emulsion, comprising the combination of an aqueous phase and an oil phase, wherein the aqueous phase comprises clay particles; and wherein the oil phase comprises tetraalkyl orthosilicate.

Advantageously, the oil phase comprises an active ingredient.

This method produces mechanically strong microcapsules through the formation of a clay-silica composite wall via interfacial hydrolysis of the tetraalkyl silicate. By ‘mechanically strong’ we mean that the wall is sufficiently strong such that the capsules remain intact upon dry down of the wet system and/or sufficiently contiguous to give controlled release of any active ingredient from within the capsule.

The Pickering emulsion may be formed by high shear incorporation of the oil phase into an aqueous external phase comprised of an aqueous dispersion of the clay particles in water. The process is preferably carried out at 20 to 80° C., such as from 30 to 60° C., from 40 to 59° C., or most preferably from 45 to 50° C.

Preferably the oil phase is present in the emulsion in an amount from 5 to 60%, more preferably from 10 to 50%, or from 15 to 40%, by weight based on the total mass of emulsion.

Any active ingredient encapsulated within the core of the microcapsules is suitably less than 10 percent by weight soluble in water and more suitably less than 1 percent by weight soluble in water; and most suitably less than 0.1 percent by weight soluble in water.

A wide range of active materials (active ingredients) may be encapsulated including inks, flavours, cosmetics, perfumes, sunscreens, fragrances, adhesives, sealants, phase change materials, biocides, oilfield chemicals (including corrosion and scale inhibitors), flame retardants, food additives (including vitamins, ingredients, probiotics and antioxidants), active agents that may be included in detergent, fabric softeners and other household products (such as bleaches, enzymes and surfactants), active agents that may be included in textiles (such as insect repellents, antimicrobial agents, skin softeners and medically active compounds), active agents that may be included in coatings (such as fire retardant, flame retardant, antifouling, antibacterial, biocidal, scratch resistant and abrasion resistant compounds) and biologically active compounds (such as pharmaceuticals and agrochemicals). Suitably the active material is an agrochemical such as a herbicide, fungicide or insecticide. Many such agrochemicals are known and are described in The Pesticide Manual 14th edition published by the British Crop Protection Council in 2006. The invention is also suitable for encapsulating a solid complex of an agrochemical with a molecular complexing agent including, for example, a complex of 1-MCP and a-cyclodextrin. The invention is most useful for agrochemicals that are subject to degradation when exposed to sunlight, in particular pyrethroid insecticides such deltamethrin, tralomethrieta, cyfluthrin, alphamethrin, zeta-cypermethrin, fenvalerate, esfenvalerate, acrinathrin, allethrin, bifenthrin, bioallethrin, bioresmethrin, cycloprothrin, beta-cyfluthrin, cyhalothrin, beta-cypermethrin, cyphenothrin, empenthrin, etofenprox, fenpropathrin, fiucythrinate, tau-fluvalinate, phenothrin, prallethrin, resmethrin, tefluthrin, tetramethrin, and lambda-cyhalothrin; suitably lambda-cyhalothrin. Suitably, microcapsules of the present invention may be used in wallboards or plasterboards in buildings, and may be used in improving cement compositions and processes for making cementitious materials.

The active ingredient is suitably a pharmaceutical compound or an agrochemical; most suitably it is an agrochemical.

Suitably, the agrochemical is a fungicide, insecticide, herbicide or growth regulator, used for controlling or combating pests such as fungi, insects and weeds or for controlling the growth of useful plants. The agrochemical may also be used in non-agricultural situations (for example public health and professional product purposes, such as termite barriers, mosquito nets and wallboards).

Further suitable applications include, without limitation:

Sustained release or controlled release usages, for example: pharma, for example acid resistant capsules (oral delivery past low pH in the stomach), protection of labile actives, pseudo-zero order release through capsule wall and Ostwald-ripening resistant emulsion formulations; cosmetics; perfumes, for example slowing down evaporation of top-notes or sustained release and minimising overpowering odours; capsules having affinity for cellulose and trapped on textile surface during laundering; flavours, for example light stabilised to prevent oxidation; self-healing coatings, for example capsule bursts to release a resin that repairs damage; carbonless copy paper; novel, double taste and texture food, for example capsule which dissolves in the mouth and releases a new taste; pressure sensitive adhesives; sealants; nutrition (for example increased bioavailability of complex molecules and protection of sensitive molecules such as vitamins, probiotics and other food additives); toner inks with photosensitivity or thermal sensitivity; textile coatings, for example, for improving permeability properties; antifouling coatings; surface protective coatings, for example, for improving scratch or abrasion resistance; and construction materials, for example wall-boards, plasterboards and cements. Example of capsules that are dried out, include, for example, various mineral blends to form a ceramic upon calcination; low density fillers for polymers or paints; insulating materials; low density proppants; light reinforcing particles, for example for wood-fibre composites; recyclable pigments, for example low density allowing easy flotation separation; and energy buffers, for example use in a void in spheres to provide a ‘crash barrier’ with adsorption of energy. Capsules of the present invention may be of novel size or shape, for example: creation of plate or rod shape capsules; and use of metallic particles resulting in conductive capsules, or having a metallic nature, for example plasmon absorbance.

Advantageously, the oil phase comprises an alkanoic acid, preferably a primary carboxylic acid. Preferably, the primary carboxylic acid with an alkyl chain of from 4 to 10 carbons, more preferably from 6 to 8 and most preferably 8 (i.e., n-octanoic acid). Without wishing to be bound by theory, it is believed that the presence of the alkanoic acid advantageously delivers the proton ion in situ at the optimum location and thus facilitates the formation of the microcapsule wall.

Preferably the alkanoic acid is present at a concentration of from 1 to 20% in the oil phase based on the mass of the oil phase, preferably from 5 to 15% by weight and most preferably from 9 to 11% (such as 10%) by weight.

Advantageously, the oil phase comprises an alkyl sulphonic acid and/or alkyl aryl sulphonic acids.

The clay particles may be kaolin clay. Kaolin clay is also referred to as china clay or hydrous kaolin and is predominantly mineral kaolinite (Al₂Si₂O₅(OH)₄), a hydrous aluminium silicate (or aluminosilicate). Preferably the clay particles are present in the aqueous phase in an amount of from 1 to 20% by weight, more preferably from 2 to 15% by weight.

The microcapsules preferably have a volume mean droplet diameter, assessed by laser diffraction, in the size range of from 0.5 to 100 μm, preferably from 1 to 50 μm.

The clay particles suitably have a particle size distribution wherein the median diameter (d₅₀) is less than or equal to 10 μm, as measured by determining the sedimentation speeds of the dispersed particles of the particulate material under test through a standard dilute aqueous suspension using a SEDIGRAPH™, for example SEDIGRAPH™ 5100, obtained from Micromeritics Corporation, USA. Suitably, the particulate inorganic material has a d₅₀ less than or equal to 5 μm. More suitably, the particulate inorganic material has a d₅₀ less than or equal to 2 μm. Yet more suitably, the particulate inorganic material has a d₅₀ less than or equal to 1 μm. In increasing suitability, the particulate inorganic material has a d₅₀ less than or equal to 0.9, 0.8, 0.7, 0.6, 0.5, 0.4, or 0.3 μm. In other aspects, the particulate inorganic material has a d₅₀ less than or equal to 0.2 μm, for example less than or equal to 0.15 μm or less than or equal to 0.12 μm or less than or equal to 0.1 μm.

In one aspect, at least about 90 percent of the particles of the particulate inorganic material by weight are smaller than about 2 μm, for example at least about 95 percent or 98 percent are smaller than about 2 μm. Suitably, at least about 90 percent of the particles by weight are smaller than about 1 μm, for example at least about 95 percent or 98 percent are smaller than about 1 μm. More suitably, at least about 75 percent of the particles by weight are smaller than about 0.25 μm, for example at least about 80 percent or 82 percent are smaller than about 0.25 μm. In another aspect, the particulate inorganic material has a particle size distribution of (i) at least about 90 percent of the particles by weight less than about 2 μm, for example at least about 95 percent or 98 percent; (ii) at least about 90 percent of the particles by weight are less than about 1 μm, for example at least about 95 percent or 98 percent; and (iii) at least about 75 percent of the particles by weight are less than about 0.25 μm, for example at least about 80 percent or 82 percent; and particulate inorganic material of such particle size distributions may also have d₅₀ values at the smaller end of the range, for example at least about 98 percent of the particulate inorganic material by weight is smaller than about 2 μm, at least about 98 percent is smaller than about 1 μm, at least about 82 percent is smaller than about 0.25 μm, and the d₅₀ value of the particulate inorganic material is less than or equal to 0.12 μm.

For finer particulate inorganic material (for example having a d₅₀ less than or equal to 2 μm), the material may be derived through classification, including methods such as gravity sedimentation or elutriation, use of any type of hydrocyclone apparatus or, for example, a solid bowl decanter centrifuge or a, disc nozzle centrifuge. The classified particulate inorganic material may be dewatered in one of the ways known in the art, for example filtration (including filter press), centrifugation or evaporation. The classified, dewatered material may then be thermally dried (for example, by spray drying).

Preferably the clay particles have been surface-modified to have a population of positively charged sites. These positively charged sites attract the negative ions produced during the hydrolysis of the silicate and thus promote the formation of the composite wall. It should be noted that the addition of positively charged sites relative to the unmodified particles may not necessarily lead to an overall positive charge for the material, but may do so depending on the level of modification.

The clay particles may be surface-modified with an amino-silane, preferably aminopropyl triethoxysilicate. The silane groups react with the clay so as to give free amine groups attached to the clay surface.

Alternatively, the clay particles may be surface-modified through the incorporation of a cationic surfactant, such as alkyl trimethyl ammonium bromide, most preferably dodecyl trimethyl ammonium bromide. The addition of the cationic surfactant may take place either pre or post emulsification of the oil phase. The cationic surfactant is present in a concentration of from 0.01 to 2% by weight, such as 0.05 to 1% by weight.

The aqueous phase may comprise an electrolyte, such as sodium chloride, sodium sulphate, potassium sulphate, magnesium chloride or magnesium sulphate. The electrolyte is in the concentration range of from 0 to 25% by weight of the aqueous phase, such as from 1 to 15% by weight, from 2 to 13% by weight.

Preferably the alkyl chain of the tetraalkyl orthosilicate contains from one to four carbons, preferably three carbons (i.e., tetrapropyl orthosilicate). The tetraalkyl orthosilicate may be present at a concentration of from 1 to 10% by weight in the oil phase, such as from 2 to 9% by weight, from 3 to 8% by weight, or most preferably from 4 to 6% (e.g., 5%) by weight.

Surprisingly the encapsulation method is effective in an intermediate pH range of 3.5 to 6, preferably from 4 to 5.5. Silica wall formation in the art is normally carried out at more extreme pH values (either highly acidic or highly alkaline) but the moderate pH described herein is both effective in microcapsule formation and may reduce any breakdown of active ingredients that are sensitive high or low pHs. The pH may be adjusted through the addition of a mineral acid or base in the aqueous phase.

In a second aspect of the invention there is provided a composition comprising one or more microcapsules in an aqueous phase, wherein the microcapsule(s) comprise a hydrophobic liquid and an inorganic microcapsule wall, and wherein the microcapsules are prepared by a process as described herein.

Advantageously the hydrophobic liquid comprises an active ingredient, preferably an agrochemical. Advantageously the microcapsules exhibit controlled release of the active ingredient.

Preferably the inorganic microcapsule wall comprises clay particles and/or silicates. Advantageously the clay particles have been surface-modified to have a population of positively charged sites. Preferably the inorganic microcapsule wall does not contain an organic cross-linker.

In a third aspect of the invention there is provided the use of a composition as described herein as an agrochemical composition for controlling plants, pests and/or fungicides

Unless otherwise stated percentages are given as percentages by total weight and all embodiments and preferred features may be combined in any combination.

The present invention is exemplified with the examples below which are not meant to limit the scope of the invention.

### EXAMPLES

### Example 1

### Emulsification Method

Dispersions of clay particles in water were prepared by adding the required amount of clay (5 g) to water (95 g giving a solids concentration of 5% w/w). The mixture was subjected to ultrasonic agitation using a high intensity probe set to a 5% work cycle in which 1 s agitation was followed by 1 s rest for a total time of 2 minutes. This was found to result in the complete dispersion of the particles in the aqueous phase.

Oil phases were prepared by mixing together the required components to form a homogenous solution. The oil phase was added to the aqueous phase typically in a mass ratio of 10:90 g or 20:80 respectively. The mix was shaken by hand to pre-mix the oil phase and then subjected to high shear mixing using a rotor stator system (Ystral®, 10 mm head at a speed of 15000-20000 rpm for 5 to 10 minutes) to form a Pickering emulsion of oil droplets stabilised by clay particles adsorbed at the O/W interface.

The droplet size distributions of the oil droplets were then determined using laser diffraction (Malvern 2000 Laser Diffraction Particle Sizer) to give the volume mean diameter (VMD).

### Example 2

An emulsion was prepared as described in Example 1, containing 20% w/w oil phase:

Oil phase: 40% Solvesso 200nd (Exxon), 40% Lambda cyhalothrin, 10% tetrapropyl orthosilicate 10% n-octanoic acid;

Aqueous phase: 5% aminopropyl modified kaolin (RLO7645, ex. Imerys).

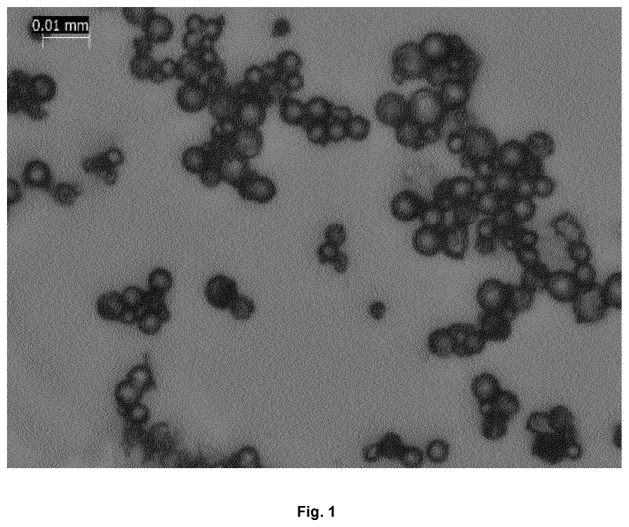

The emulsion was stored under quiescent conditions at 50° C. After storing for 1 week the samples were allowed to dry down on microscope slides and their appearance determined under a microscope. The appearance is shown in FIG. 1 and demonstrates that a mechanically strong composite silica/kaolin wall had formed.

The rate of release of the lambda cyhalothrin from the sample (VMD 14.8 μm) was determined by diluting the capsule suspension in water and this suspension was placed in contact with n-hexane and rolled. At suitable time intervals the n-hexane phase was sub-sampled and analysed for lambda cyhalothrin. This was repeated for two commercial lambda cyhalothrin formulations designated as fast release (Warrior CS, VMD 2.8 μm) and slow release (Demand CS (VMD 15.8 μm)) formulations.

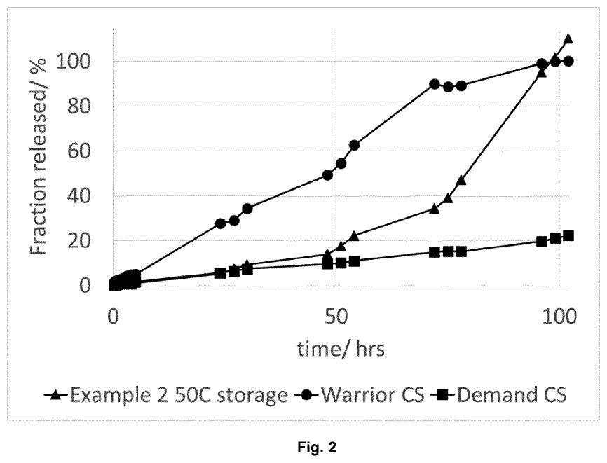

Plots of fraction of lambda cyhalothrin released (relative to the total content in the diluted suspension) were constructed and are presented in FIG. 2. The kaolin/silica capsule showed a release profile intermediate between the designated fast and slow-release formulations indicating that the composition could give comparable release rates to current commercial formulations.

### Example 3

Three emulsions were prepared using the method given in Example 1 in which the oil phase content was varied from 10 to 30% w/w in order to identify optimum oil phase content.

Oil phase: 70% Solvesso 200nd, 10% dimethyl phthalate, 10% tetrapropyl orthosilicate, 10% n-octanoic acid

Aqueous phases: 6%, 5% and 4% w/w aminopropyl modified kaolin (RLO7645, ex. Imerys) for 30, 20 and 10% w/w oil content respectively.

The pHs of the three emulsions (containing 10, 20 and 30% organic phase) were adjusted to pH 4.5-5 and then stored at 50° C. under quiescent conditions for 3 days and their mechanical strengths on dry down on a microscope slide were evaluated under the microscope.

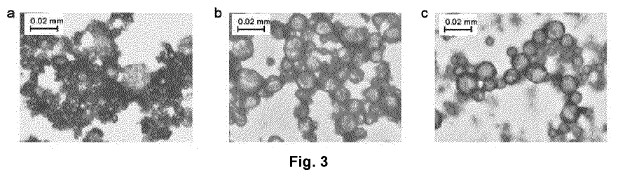

FIG. 3 shows the dried down samples of Example 3: (a) 10% w/w oil phase, (b) 20% oil phase (c) 30% Oil phase; after storage for 3 days at 50° C.

The samples containing 20 and 30% oil phase showed capsules with improved mechanical strength that remained unbroken upon dry down.

### Example 4

These experiments demonstrated the improved robustness of the capsule formation in the presence of added sodium chloride for systems containing lambda cyhalothrin.

An emulsion was prepared as described in Example 1 containing 20% w/w oil phase comprised 10% n-octanoic acid and 10% tetrapropyl orthosilicate, 40% lambda cyhalothrin in Solvesso 200nd.

The aqueous phase comprised 5% aminopropyl modified kaolin (RLO7645, ex. Imerys) or 5% w/w kaolinite (ex. Sigma Aldrich) each with 0 and 1500 mM sodium chloride. The emulsion systems were stored at 50° C. for 6 days prior to evaluation. Incorporation of the sodium chloride improved the formation of mechanically strong capsules with both the modified and the unmodified kaolin

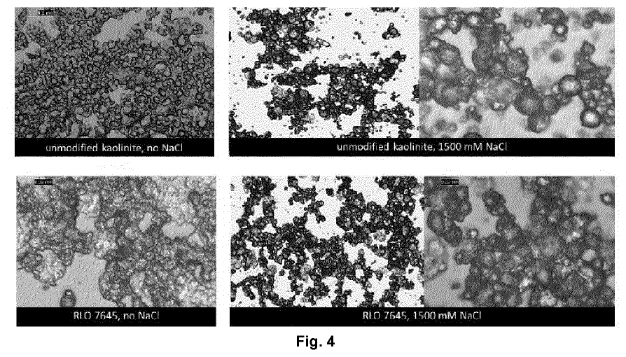

FIG. 4 shows the dry down structures formed by emulsions stored at 50° C. for 6 days and stabilised by modified and unmodified kaolin in the absence and presence of added sodium chloride. The Figure demonstrates that the incorporation of sodium chloride further improved the formation of mechanically strong structures.

### Example 5

These experiments demonstrated the improved robustness of the capsule formation in the presence of further added electrolytes for systems containing lambda cyhalothrin.

An emulsion was prepared as described in Example 1, containing 20% w/w oil phase comprised 10% n-octanoic acid and 10% tetrapropyl orthosilicate, 40% lambda cyhalothrin in Solvesso 200nd.

The aqueous phase comprised 5% aminopropyl modified kaolin (RLO7645, ex. Imerys) or 5% w/w kaolinite (ex. Sigma Aldrich) both in the absence of added electrolyte and in the presence of the following electrolytes. The pH of the emulsion system was in the range 4.5-5.

The following electrolytes were used:
500 mM Potassium sulphate
500 mM Sodium sulphate
375 mM Magnesium sulphate
500 mM Magnesium chloride

The presence of the added electrolyte had a generally small effect on the measured droplet size (volume mean diameter, shown in Table 1) showing that the systems were not being grossly flocculated by the salt.

TABLE 1

| | Volume mean diameter/microns | | | | |
|---|---|---|---|---|---|
| | | | | 375 mM | 500 mM |
| | | 500 mM | 500 mM | Magne- | Magne- |
| | No | Potassium | Sodium | sium | sium |
| | additive | sulphate | sulphate | sulphate | chloride |
| Unmodified | 18.6 | 19.7 | 29.7 | 21.6 | 18.8 |
| kaolin | | | | | |
| RLO7645 | 14.3 | 18.3 | 15.8 | 14.3 | 17.6 |

The emulsions were stored quiescently for 10 days and in the absence of added electrolyte the systems formed from both the aminopropyl silane modified (RLO7645) and the unmodified kaolins showed breakdown of the droplets on dry down.

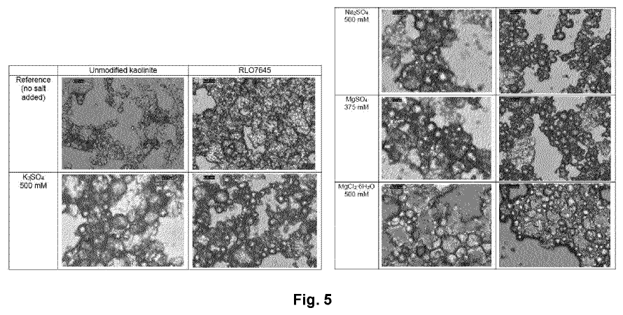

Incorporation of the electrolytes produced mechanically strong capsules on dry down on a microscope slide with both the modified and the unmodified kaolin. FIG. 5 shows the dry down structures formed by capsule formulations stabilised by modified and unmodified kaolin in the absence and presence of added electrolytes. The image shows that the incorporation of the electrolytes improved the process and gave mechanically strong structures on dry down on a microscope slide after 10 days storage at 50° C.

### Example 6

This experiment shows that robust capsules could be robustly made by ensuring the level of surface amination.

Both previously unmodified kaolin (ex. Sigma Aldrich) and the modified kaolin (RLO7645) were subjected to further amination with aminopropyl triethoxy silane (APTES). An initial experiment in which both of two the kaolin samples were further modified by placing 20 g of the kaolin in 100 g of a 5% solution of APTES in dichloromethane was found to over modify the surface and emulsion formation was relative to pH. The degree of modification was decreased by reducing the concentration of APTES in the DCM. The RLO7645 was treated by adding 20 g of the kaolin to 100 g of 0.5, 1 and 2.5 w/w APTES in DCM. After 1 day the excess liquid was decanted off and the clay allowed to dry.

The modification of the kaolin was demonstrated by determining the zeta potential of the clay as a function of pH using a Malvern Zetasizer. Treatment of the RLO7645 increased the iso-electric point (pH where the zeta potential is zero) from pH 4.4 to pHs in excess of 5.3 with the 5% APTES treatment giving an isoelectric point of pH9.4

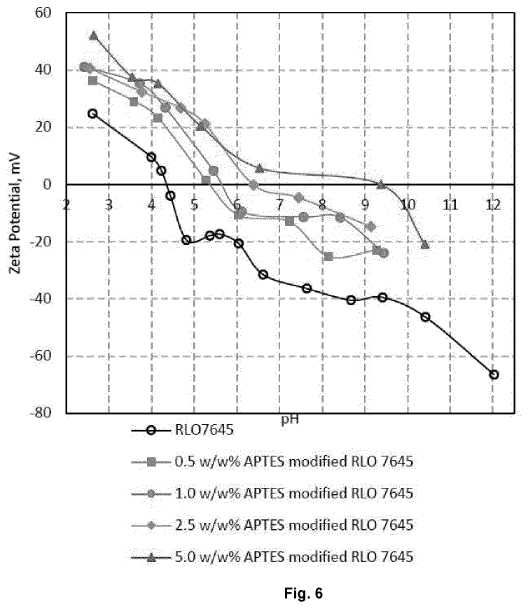

FIG. 6 demonstrates the Zeta potential as a function of pH for unmodified and modified RLO7645.

Emulsions containing 20% w/w oil phase comprised of 10% n-octanoic acid, 10% tetrapropyl orthosilicate, 40% lambda cyhalothrin in a Solvesso 200nd carrier were prepared as described in Example 1 and stored at 50° C. The aqueous phase comprised 5% w/w of the 0.5, 1 and 2.5% APTES modified RLO7645.

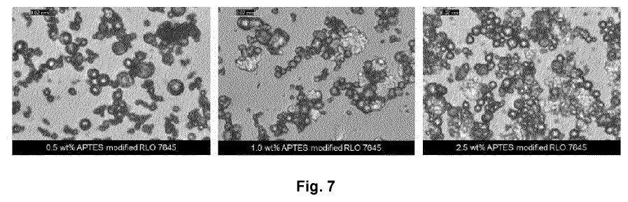

Mechanically strong capsules were seen upon dry down and are shown in FIG. 7. The formulation on a microscope slide demonstrating that the level of amination is a key parameter in the formation of stable capsule formulations.

### Example 7

These experiments show the benefit of addition of a cationic surfactant to the aqueous phase, either prior to emulsification of the oil into the aqueous phase or as a post addition after emulsification.

Two emulsions were prepared following the general method outlined in Example 1, with an organic phase comprised of 40% w/w Lambda cyhalothrin, 10% w/w n-octanoic acid, 10% w/w tetrapropyl orthosilicate.

In the first emulsion the aqueous phase was comprised 5% w/w RLO7645 and after the emulsion had been formed, dodecyl trimethyl ammonium bromide was added to give a concentration of 0.2% to the aqueous phase.

In the second the aqueous comprised 5% w/w RLO7645 and 0.2% w/w of the surfactant dodecyl trimethyl ammonium bromide prior to emulsification.

The two systems were stored at 50° C. for 6 days and evaluated for capsule mechanical strength by dry down on microscope slides. Both systems produced mechanically strong capsules on drying in the presence of the DTAB which was adsorbing on the clay surface increasing the positive charge on the surface

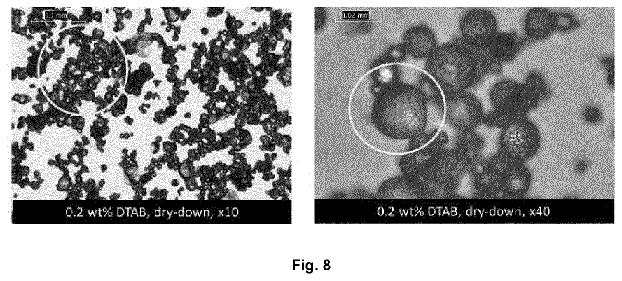

FIG. 8 shows the dry down views of mechanically strong capsules formed in the presence of DTAB added to the aqueous phase prior to emulsification and stored for 6 days at 50° C.

The invention is defined by the claims.

1. A method of forming a Pickering emulsion, comprising the combination of an aqueous phase and an oil phase,
wherein the aqueous phase comprises clay particles; and
wherein the oil phase comprises tetraalkyl orthosilicate.

2. A method according to claim 1, wherein the oil phase comprises an active ingredient, preferably an agrochemical.

3. A method according to claim 1, wherein the oil phase comprises an alkanoic acid, preferably a primary carboxylic acid.

4. A method according to claim 1, wherein the clay particles have been surface-modified to have a population of positively charged sites.

5. A method according to claim 4, wherein the clay particles have been surface-modified with an amino-silane.

6. A method according to claim 4, wherein the clay particles have been surface-modified through the incorporation of a cationic surfactant.

7. A method according to claim 1, wherein the aqueous phase comprises an electrolyte.

8. A method according to claim 1, wherein the alkyl chain of the tetraalkyl orthosilicate contains from one to four carbons, preferably 3 carbons.

9. A method according to claim 1 that is carried out at a pH of from 3.5 to 6, preferably from 4 to 5.5.

10. A composition comprising one or more microcapsules in an aqueous phase, wherein the microcapsule(s) comprise a hydrophobic liquid and an inorganic microcapsule wall, and wherein the microcapsules are formed by a method according to claim 1.

11. A composition according to claim 10, wherein the hydrophobic liquid comprises an active ingredient, preferably an agrochemical.

12. A composition according to claim 10, wherein the inorganic microcapsule wall comprises clay particles and/or silicates.

13. A composition according to claim 12, wherein the clay particles have been surface-modified to have a population of positively charged sites.

14. A composition according to claim 13, wherein the inorganic microcapsule wall does not contain an organic cross-linker.

15. A composition according to claim 11, wherein the microcapsules exhibit controlled release of the active ingredient.

16. Use of a composition according to claim 11 as an agrochemical composition for controlling plants, pests and/or fungicides.
